(19) United States
(12) Patent Application Publication
(10) Pub. No.: US 2023/0218184 A1
(43) Pub. Date: Jul. 13, 2023

(54) NARROW BEAM OPTICAL SENSOR SYSTEM

(71) Applicant: Maxim Integrated Products, Inc., San Jose, CA (US)

(72) Inventors: Judy Lau Hermann, Palo Alto, CA (US); Sudhir Mulpuru, Milpitas, CA (US); Chung-Che Huang, Cupertino, CA (US)

(21) Appl. No.: 17/481,208

(22) Filed: Sep. 21, 2021

Related U.S. Application Data
(60) Provisional application No. 63/081,145, filed on Sep. 21, 2020.

(51) Int. Cl. A61B 5/024

(52) U.S. Cl. CPC A61B 5/02427; A61B 2562/12

(57) ABSTRACT

An optical sensor system and method of manufacture thereof can include: providing a sensor body; mounting a light emitter to the sensor body, the light emitter for emitting light into tissue; mounting a light detector to the sensor body for providing a physiological measurement from within the tissue; and affixing an optical film above the light detector, the light being angularly constrained by the optical film.

### CROSS-REFERENCE TO RELATED APPLICATION(S)

This claims priority benefit to all common subject matter of U.S. Provisional Pat. Application 63/081,145 filed Sep. 21, 2020. The content of this application is incorporated herein by reference.

### TECHNICAL FIELD

This disclosure relates to optical biological sensors; more particularly, to optical sensors utilizing narrow beam optical signals providing deep penetrating measurements.

### BACKGROUND

The rapidly growing market for portable and wearable electronic devices represents one of the largest potential market opportunities for next generation biological sensors. These devices have unique attributes that have significant impacts on their design and manufacture, in that they must be generally small, lightweight, and rich in functionality, and they must be produced in high volumes at relatively low cost.

One extension of the portable and wearable electronics industry is the biological sensor industry, which includes optical sensors for measuring heart rate, blood pressure, peripheral oxygen, and other physiological readings for example. The biological sensor industry, similar to the portable and wearable electronics industry, has witnessed ever-increasing commercial competitive pressures, along with growing consumer expectations and the diminishing opportunities for meaningful product differentiation in the marketplace.

Sensor system size, sensor system design, and sensor system materials are evolving technologies at the very core of next generation biological sensors. These next generation biological sensors, likewise, are outlined in road maps for development of next generation consumer products.

Importantly, as sensor systems are implemented in smaller form factors, including rings and jewelry, sensor footprint becomes an increasingly primary and overriding design constraint. Together with sensor footprint, sensor performance increasingly weighs as a primary design constraint.

One key to improving the sensor performance is to ensure light, emitted into the tissue being measured, travels through enough blood-containing-tissue to get a proper reading. One prior solution focused on increasing the separation distance between a light sensor and a light emitter. Increased separation distance between a light emitter and a light sensor had the benefit of increasing the proportion of detected signal reaching deeper into the skin where the blood, and useful signal, is measured.

The sensor performance benefits obtained by increasing the separation distance between the light emitter and light detector, however, comes at the cost of a larger sensor footprint. This previous solution, disadvantageously tied performance to a larger footprint often constraining the performance of bio-sensing wearables by how far apart the light emitter and light detector are placed.

In some cases, measurements within the ear canal for example, size constraints all but prevented this previous solution from obtaining measurements with a reasonable signal to noise ratio. Many of these problems result from light traveling through shallow areas of tissue which causes larger amounts of noise, degrading the overall signal to noise ratio.

Another prior solution focused on increasing the signal to noise ratio by increasing power consumption of the light emitting element. While this prior solution allowed reliable optical data to be obtained, the substantial increase in system power consumption led to other complexities, like heat, that compromise the ability to reliably gather data. Furthermore, the substantial increase in system power consumption led to size constraints of the power storage mechanism.

In view of the ever-increasing commercial, technological, and consumer expectations and the diminishing opportunities for meaningful product differentiation in the marketplace, it is critical that systems providing smaller footprints with enhanced sensing performance be developed to provide measurement performance solutions that are not tied to increasing the separation distance between a light sensor and a light emitter. Solutions to these problems have been long sought, but prior developments have not taught or suggested any complete solutions and, thus, solutions to these problems have long eluded those skilled in the art.

### BRIEF DESCRIPTION OF THE DRAWINGS

The narrow beam optical sensor system is illustrated in the figures of the accompanying drawings which are meant to be exemplary and not limiting, in which like reference numerals are intended to refer to like components, and in which:

FIG. 1 is a top view of the optical sensor system in a first embodiment.

FIG. 2 is a cross-sectional view of the optical sensor system along the line 2–2 of FIG. 1.

FIG. 3 is a cross-sectional view of the film of FIG. 2.

FIG. 4 is a graphical view of light transmission through the film of FIG. 2.

FIG. 5 is a control flow for the manufacture of the optical sensor system of FIG. 1.

FIG. 6 is a cross-sectional view of the optical sensor system in a second embodiment.

FIG. 7 is a cross-sectional view of the light emitter of FIG. 6.

FIG. 8 is a control flow for the manufacture of the optical sensor system of FIG. 6.

### DETAILED DESCRIPTION

In the following description, reference is made to the accompanying drawings that form a part hereof, and in which are shown by way of illustration, embodiments in which the optical sensor system may be practiced. It is to be understood that other embodiments may be utilized and structural changes may be made without departing from the scope of the optical sensor system.

When features, aspects, or embodiments of the optical sensor system are described in terms of steps of a process, an operation, a control flow, or a flow chart, it is to be understood that the steps can be combined, performed in a different order, deleted, or include additional steps without departing from the optical sensor system as described herein.

The optical sensor system is described in sufficient detail to enable those skilled in the art to make and use the optical sensor system and provide numerous specific details to give a thorough understanding of the optical sensor system; however, it will be apparent that the optical sensor system may be practiced without these specific details.

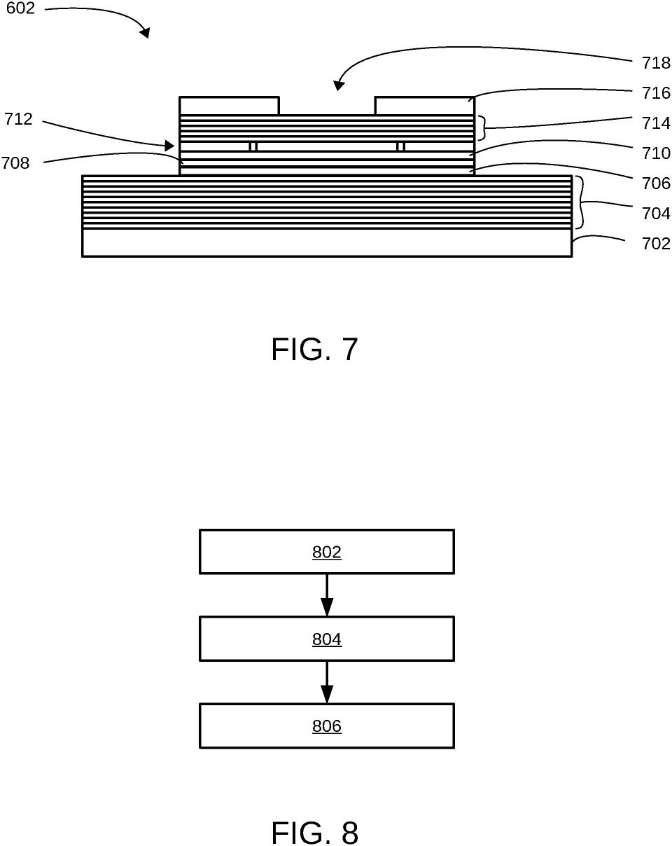

In order to avoid obscuring the optical sensor system, some well-known system configurations and descriptions are not disclosed in detail. For example, the light emitter of FIG. 7 together with the tissue of FIGS. 2 and 6 are shown simplified for clarity. Likewise, the drawings showing embodiments of the system are semi-diagrammatic and not to scale and, particularly, some of the dimensions are for the clarity of presentation and are shown greatly exaggerated in the drawing FIGs.

As used herein, the term system is defined as a device or method depending on the context in which it is used. For expository purposes, the term “horizontal” as used herein is defined as a plane parallel to the top plane or surface of the light detector, regardless of its orientation. The term “vertical” refers to a direction perpendicular to the horizontal as just defined. Terms, such as “above”, “below”, “bottom”, “top”, “side”, “higher”, “lower”, “upper”, “over”, and “under”, are defined with respect to the horizontal plane.

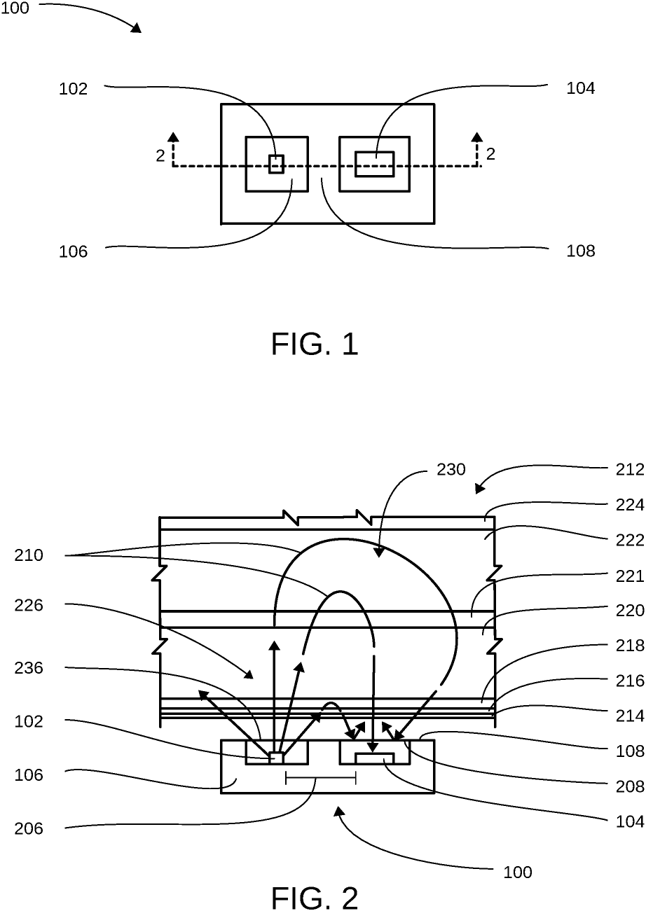

Referring now to FIG. 1, therein is shown a top view of the optical sensor system 100 in a first embodiment. The optical sensor system 100 is shown having a light emitter 102 positioned next to and offset from a light detector 104. The light emitter 102 and the light detector 104 can be mounted to a sensor body 106 between vertically extended sidewalls 108.

Referring now to FIG. 2, therein is shown a cross-sectional view of the optical sensor system 100 along the line 2–2 of FIG. 1. The optical sensor system 100 is shown having the light emitter 102 and the light detector 104 of FIG. 1.

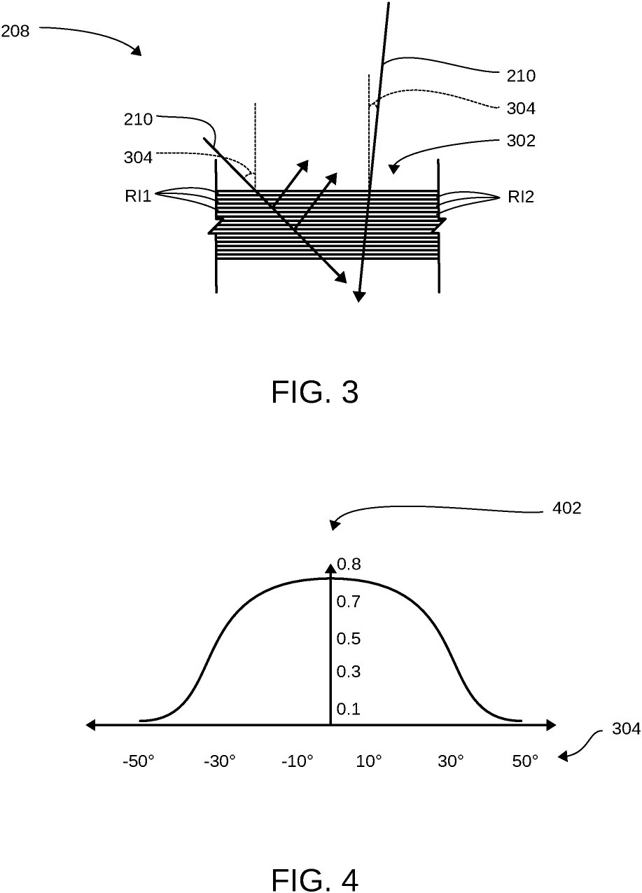

The light detector 104 and the light emitter 102 can be positioned next to one another with a separation distance 206, therebetween. The separation distance 206 can be made very small, for example by utilizing a multi-layer optical film (MOF) or film 208, as shown and described with regard to FIG. 3.

For descriptive clarity, the film 208 is to be understood as an angular filter and can include horizontal optical layers of alternating refractive indices. The horizontal optical layers of alternating refractive indices can provide an angular constraint on the light 210 generating a narrow beam incident on the light detector 104; because, as the light 210 increasingly deviates from a vertical or perpendicular incidence on the film 208 it is increasingly reflected by the film 208.

The light 210 being angularly constrained by the film 208 can thereby provide a narrow beam light path perpendicular to the light detector 104 decreasing signal to noise ratio by filtering out the shallow and noisier signals. The light emitter 102 can be a light emitting diode (LED) emitting light from a top surface and sides of the light emitter 102. The light detector 104 can be a photodiode. The light emitter 102 can emit the light 210 into a tissue 212 of a person or an animal.

The light 210 can be reflected or back-scattered from within the tissue and be incident on the light detector 104, where the light 210 is measured. As is common in the field of optical physiological measurements, the light 210 that is back-scattered and absorbed within the tissue 212 will be absorbed and back-scattered based on oxygen and other components of fluids within the tissue 212.

The light detector 204 can output a changing voltage based on the change in the light 210 absorbed and back-scattered due to respiration, heart rate, and other processes known as a photoplethysmograph (PPG). Although the PPG signals are described as relating to heart rate and respiration, other frequency components of the PPG signal are contemplated as well.

For descriptive clarity, the tissue 212 is described using simplified terms and a seven-layer skin model, which is simplified by stratifying the tissue matrix into multiple layers. Other skin structures are contemplated to be compatible with the optical sensor system 100 as the filtering properties of the film 208 can be shared across differing biologies. The tissue can include a stratum corneum 214, an epidermis 216, a papillary dermis 218, a superior blood net dermis 220, a reticular dermis 221, an inferior blood net dermis 222, and subcutaneous fat 224.

The light 210 back-scattered by the stratum corneum 214, the epidermis 216, the papillary dermis 218, the superior blood net dermis 220, and the reticular dermis 221 increase the noise of any reflected light and also provide little appreciable measurable volumes. The light 210 emitted into, and back-scattered by the inferior blood net dermis 222 can provide valuable measurements of volumetric blood changes.

The inferior blood net dermis 222, also known as the cutaneous plexus, can provide better measurements than shallower layers as blood is more present in the inferior blood net dermis 222. It has been discovered that utilizing the film 208 implemented as an angular filter using multiple layers of alternating refractive indexes allows the light 210 to be reflected when the light 210 takes a shallow path through the stratum corneum 214, the epidermis 216, the papillary dermis 218 the superior blood net dermis 220, and the reticular dermis 221.

This provides a narrow beam of the light 210 from within the tissue 212 and the light 210 with the largest amount of noise and the least amount of measurable traits is reflected by the film 208 as it has a large deviation from vertical. The angularly constrained signal is therefore a narrow beam with respect to the light detector 104, in that the light 210 incident on the light detector 104 is largely perpendicular or having a deviation of less than 30° from perpendicular. As is shown with respect to FIG. 4, nearly half of the light 210 is reflected off of the film 208 when the angle is 30° from the vertical, and the reflection rate increases to over 90% for the light 210 with an angle of over 50° from the vertical.

The narrow beam being angularly constrained therefore leads to a better overall detected signal without increasing power or increasing the separation distance 206. The light 210 with an angle of incidence that deviates from the vertical is shown reflected off of the film 208, while the light 210 with an angle of incidence perpendicular to the film 208 is shown traversing through the film 208 and incident on the light detector 104.

More particularly, the light 210 is shown incident on the film 208, from the left, the right, and the top. The light 210 that strikes the film 208 at an angle, from the left and the right, is shown reflected away from the light detector 104 and thus angularly constrains the signal and effectively provides a narrow beam detected by the light detector 104.

The light 210 is therefore angularly constrained by the film 208 to a light path having a narrow beam and perpendicular to the light detector, as light that is not perpendicular to the film 208 will be increasingly reflected. The light 210 perpendicularly incident on the film 208 is to be understood as a narrow beam perpendicularly incident on the light detector 104 once the light 210 passes through the filter 208. The perpendicular nature of the light path 230 comes from the light 210 emitted from the light emitter 102 being back-scattered and absorbed from deep within the tissue 212, rather than shallower near the light emitter 102.

Furthermore, the light 210 that is back-scattered within the inferior blood net dermis 222 is shown to take a longer path up through the tissue 212, and is shown substantially perpendicular to the film 208. The light 210 back-scattered within the inferior blood net dermis 222 and perpendicular to the film 208 is shown to pass through the film 208 to be incident on the light detector 104.

The light emitter 102, implemented as an LED, can provide a wide radiation pattern 226 that is emitted as the light 210 into the tissue 212. The radiation pattern 226 of the LED can be much wider than the light emitter 602 described with regard to FIG. 6.

The light 210 from the light emitter 102 is shown with a light path 230 radially extended out from the top and sides of the light emitter 102 and through the stratum corneum 214, the epidermis 216, the papillary dermis 218, the superior blood net dermis 220, and the reticular dermis 221.

This results in appreciable back-scattering of the light 210 having an angle of incidence that deviates from a vertical path, which are not perpendicular to the film 208.

It has been discovered that the film 208 implemented as an angular filter thereby provides a reflection of the light 210 back-scattered within shallower areas of the tissue 212, this effectively removes these signals having large amounts of noise and little in the way of valuable measurements. It has been further discovered that the film 208 implemented as an angular filter can allow the light 210, with an angle of incidence closer to vertical, to pass and be measured by the light detector 104.

It has been discovered that the deeper portions of the tissue 212, such as the inferior blood net dermis 222, provide better measurements as the volume of blood is larger and more active within the inferior blood net dermis 222. Measurements from deeper within the tissue 212 such as within the inferior blood net dermis 222, can therefore provide better measurements with less noise than obtaining measurements from shallower tissue layers.

The reflecting of the light 210 with angles of incidence that diverge from a vertical path perpendicular to the film 208, reduces noise without increasing power or the separation distance 206 between the light emitter 102 and the light detector 104 and thus provides an appreciable increase in performance. This is due to the film only allowing the light 210 penetrating deep within the tissue 212 at the inferior blood net dermis 222, and any back-scattering from shallower layers of the tissue 212 is not detected by the light detector 104 as the light path 230 is filtered by the film 208. The film 208 improves on prior sensor solutions because the film 208 can be used with conventional LEDs while simultaneously being very thin providing a small, cost effective performance improvement to previous solutions.

The light emitter 102 can also provide multiple different wavelengths of the light 210. For example, it is contemplated that the light emitter 102 can have a wavelength of 940 nm, 660 nm, or 530 nm. A collection of all three or any combination thereof, is also contemplated to be an array describing the light emitter 102. The optical sensor system 100 providing a 940 nm light emitter 102 can be used to provide motion data, a 660 nm light emitter 102 can be used to provide SPO2 data, and a 530 nm light emitter 102 can be used to provide heart rate data.

The light emitter 102 and the light detector 104 can be mounted to the sensor body 106 between the vertically extended sidewalls 108. The vertically extended sidewalls 108 extend vertically past the light detector 104 and vertically past the light emitter 102.

It has been discovered that the radiation pattern 226 of the light 210 can be as broad as the vertically extended sidewalls 108 allow. Illustratively, for example, the light 210 is shown having the radiation pattern 626 with over ninety degrees of spread.

A protective layer 236 shown mounted to the vertically extended sidewalls 108 and extended over the light emitter 102. The protective layer 236 can be glass, polymer, ceramic, or a combination thereof and have an optical transparency in the wavelengths at which the light emitter 102 operates. The optical sensor system 100 can be operated with the protective layer 236 or the film 208 in direct contact with the tissue 212 or can be operated with a space between the tissue 212 and the protective layer 236 and the film 208 as shown.

Referring now to FIG. 3, therein is shown a cross-sectional view of the film 208 of FIG. 2. The film 208 can be seen to have multiple layers 302 of polymer films. The polymer films can be crystalline or semi-crystalline naphthalene dicarboxylic acid polyester.

In practice, the film 208 can be polyethylene naphthalate (“PEN”), or a similar polymer. Each of the multiple layers 302 can have an average thickness of about 0.5 microns.

The multiple layers 302 can be layers having alternating refractive indexes. That is, a first set of layers having a first refractive index RI1 can be alternately stacked together with a second set of layers having a second refractive index RI2.

The film 208 is contemplated to consist of more than two layers, and is illustratively depicted having over fifteen layers. While the film 208 may contain the multiple layers 302, these layers are thin and the total thickness of the film 208 is contemplated to be less than fifty microns.

The light 210 is depicted having an angle of incidence 304. As will be appreciated, when the light 210 strikes the film 208 with the angle of incidence 304 diverging from vertical, or perpendicular from the film 208, the light 210 is reflected from within the film 208 as the light traverses the multiple layers 302. The more the light 210 diverges from vertical, the more the light 210 is reflected from within the film 208. Alternatively, when the light 210 strikes the film 208 with the angle of incidence 304 being closer to perpendicular relative to the film 208, the light passes through the multiple layers 302 and is detected by the light detector 104 of FIG. 1.

Referring now to FIG. 4, therein is shown a graphical view of light transmission through the film 208 of FIG. 2. The graphical view depicts the angle of incidence 304 along the horizontal axis together with a transmission ability 402 through the film 208.

The angle of incidence 304 is depicted as degrees of deviation from the vertical. That is, the center of the graphical view can be considered the light 210 of FIG. 2 having angle of incidence 304 vertical or perpendicular to the film 208. As the angle of incidence 304 extends to the sides of the graphical view, the angular degrees above or below the hypothetical vertical light path is described.

The transmission ability 402 along the vertical axis of the graphical view is normalized to one and depicts the partial transmission of the light 210 through the film 208 at the various angle of incidence 304 measured from vertical, which is to be considered perpendicular to a top surface of the film 208. Notably for example, the film 208 can transmit almost 80% of the light 210 when the light 210 strikes the film vertically. This is depicted as the angle of incidence 304 of 0° at the center of the graphical view.

As the angle of incidence 304 deviates from vertical, the transmission values drop. Illustratively, when the angle of incidence 304 is 30° above or below vertical, the transmission of the light 210 is reduced by almost 50%. As the angle of incidence 304 increases to 50° above or below the vertical, the transmission of the light 210 is reduced by over 90%.

For the purposes of this application a “narrow beam” due to the film 208 is to be understood as a beam traversing the film 208 having less than 50% transmission through the film 208 at angles of 40° or more divergence from the vertical with respect to the film 208 and greater than 50% transmission at angles of less than 40° divergence from the vertical with respect to the film 208. The narrow beam from the film 208 can be incident on the light detector 104.

As will be appreciated the film’s increasing reduction in the transmission ability 402 of the light 210 as the angle of incidence 304 increases from vertical provides less noise because the light 210 having the angle of incidence 304 diverging from vertical will generally pass through shallower areas of skin prone to noise and providing less informational return. Thus, the optical sensor system 100 of FIG. 1 can provide meaningful benefits without increasing footprint or power, and in some cases can provide better a signal to noise ratio with a smaller footprint and with less power.

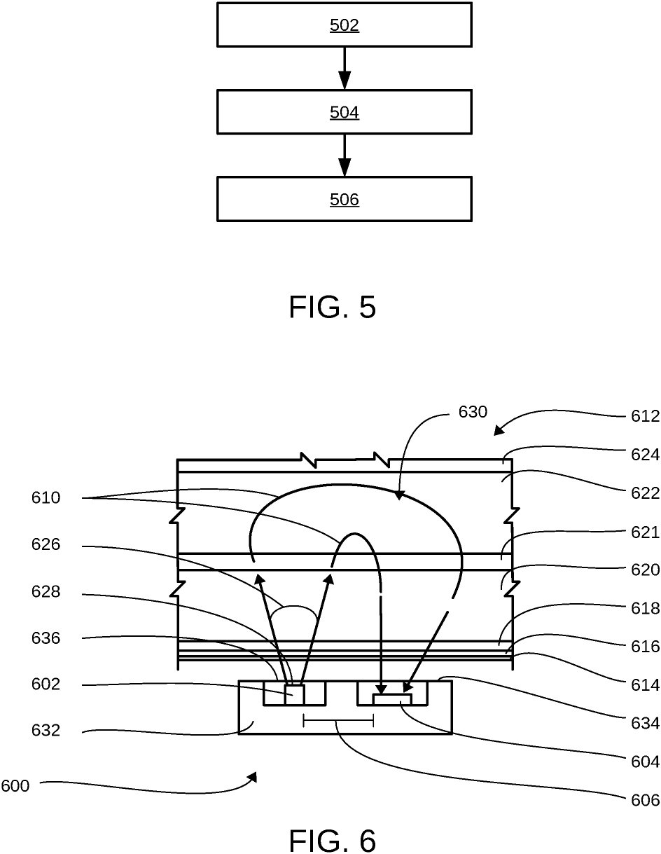

Referring now to FIG. 5, therein is shown a control flow for the manufacture of the optical sensor system 100 of FIG. 1. The method of manufacture can include providing a sensor body having a vertical extension in a block 502, mounting an LED light emitter to the sensor body, the light emitter for emitting light into tissue in a block 504; and mounting a light detector to the sensor body with the vertical extension between the light detector and the light emitter, the light detector detects the light, the light being a narrow beam and angularly constrained with an optical film affixed above the light detector for providing a physiological measurement from within the tissue in a block 506.

Referring now to FIG. 6, therein is shown a cross-sectional view of the optical sensor system 600 in a second embodiment. The optical sensor system 600 is shown having a light emitter 602 and a light detector 604.

The light detector 604 and the light emitter 602 can be positioned next to one another with a separation distance 606, therebetween. The separation distance 606 can be made very small, for example by utilizing a narrow beam emitter.

Illustratively, for example, the narrow beam could result from a vertical cavity surface emitting laser (VCSEL) as described with regard to FIGS. 7 and 8. The light detector 604 can be a photodiode.

For descriptive clarity, the light emitter 602 is set forth as a VCSEL, although it is to be understood that other laser emitters could be used such as an edge-emitting laser so long as a narrow beam is utilized. It is further contemplated that other light sources, such as LED’s, could be used when the beam is sufficiently narrowed; however as currently understood, the surface emitting laser provides the best power efficiency with a reduced cost and footprint due to the lower component count and manufacturing complexity associated with the VCSEL, for example.

The light emitter 602 can emit a narrow beam of light 610 into a tissue 612 of a person or an animal. The light 610 can be reflected or back-scattered from within the tissue and be incident on the light detector 604, where the light 610 is measured.

The light 610 from the VCSEL is shown as a narrow beam and can thereby provide a light path into the tissue 612 and back out of the tissue 612, which is perpendicular to the light detector 604. As will be appreciated, the light 610 can be back-scattered and absorbed deep within the tissue 612, and a reflection from deep within the tissue 612 will be closer to perpendicular with respect to the light detector 604, than the light 610 back-scattered from lower portions of the tissue 612.

Generating a narrow beam utilizing the light emitter 602 as a laser has therefore been discovered to decrease signal to noise ratio by eliminating shallow and noisier signals. The perpendicular nature of the light path comes from the light emitted from the light emitter 602 being back-scattered and absorbed from deep within the tissue 612, rather than being reflected shallower near the light emitter 602.

As is common in the field of optical physiological measurements, the light 610 that is back-scattered and absorbed within the tissue 612 will be absorbed and back-scattered based on oxygen and other components of fluids within the tissue 612.

The light detector 604 can output a changing voltage based on the change in the light 610 absorbed and back-scattered due to respiration, heart rate, and other processes known as a photoplethysmograph (PPG). Although the PPG signals are described as relating to heart rate and respiration, other frequency components of the PPG signal are contemplated as well.

For descriptive clarity, the tissue 612 is described using simplified terms and a seven-layer skin model, which is simplified by stratifying the tissue matrix into multiple layers. Other skin structures are contemplated to be compatible with the optical sensor system 600 as the penetrating properties of the light emitter 602 can be shared across differing biologies. The tissue can include a stratum corneum 614, an epidermis 616, a papillary dermis 618, a superior blood net dermis 620, a reticular dermis 621, an inferior blood net dermis 622, and subcutaneous fat 624.

The light 610 back-scattered by the stratum corneum 614, the epidermis 616, the papillary dermis 618, the superior blood net dermis 620, and the reticular dermis 621 increase the noise of any reflected light and also provide little appreciable measurable volumes. The light 610 emitted into, and back-scattered by the inferior blood net dermis 622 can provide valuable measurements of volumetric blood changes.

The inferior blood net dermis 622, also known as the cutaneous plexus, can provide better measurements than shallower layers as blood is more present in the inferior blood net dermis 622. It has been discovered that utilizing the light emitter 602 implemented as a laser provides a narrow beam.

More particularly, the light emitter 602 implemented as a VCSELs allows the light 610 to penetrate through the stratum corneum 614, the epidermis 616, the papillary dermis 618, the superior blood net dermis 620, and the reticular dermis 621 with little back-scattering in the direction of the light detector 604 due to the narrow radiation pattern 626 inherent in VCSEL light sources.

That is, the light emitter 602, implemented as a VCSEL, can provide a narrow beam or radiation pattern 626 with a narrow beam of 20°-30° of divergence. The light emitter 602 having a narrow beam can be the VCSEL, but could also be another type of semiconductor-based laser such as an edge-emitting laser being positioned for projecting the radiation pattern 626 into the tissue 612.

It is contemplated that the angularly constrained radiation pattern 626 having a narrow beam is due to the utilization of a laser. Some lasers are known to have a radiation pattern of nearly 40°, however many lasers, including VCSELs are known to provide less than 30° of divergence within their radiation patterns 626. For the purposes of this application, a “narrow beam” originating due to the light emitter 602 is to be understood as the radiation pattern 626 having an upper limit of 40° of divergence in a vacuum.

The light emitter 602 can be a semiconductor-based laser diode that emits the light 610 vertically only from a top surface 628 of the light emitter 602 which can beneficially provide a narrow beam. The radiation pattern 626 of the VCSEL can be much narrower than an LED which can radiate both from a top and sides of the LED. Many implementations of the VCSEL, for example, provide a radiation pattern 626 of between 19° and 27° from the top surface of the VCSEL.

The light 610 from the light emitter 602 is shown with a light path 630 vertically extended through the stratum corneum 614, the epidermis 616, the papillary dermis 618, the superior blood net dermis 620, and the reticular dermis 621. Because the light path 630 is a narrow beam, there is little appreciable back-scattering within these shallower layers of the tissue 612.

Due to the narrow beam of the light emitter 602, there is little to no light incident on the light detector 604 which was back-scattered or absorbed within these shallow layers of the tissue 612. Instead, the light 610, being a narrow beam by being emitted as a narrow laser beam, can penetrate deep within the tissue 612 where it back-scatters and is absorbed. This deep penetration within the tissue 612 provides a light path nearly perpendicular to the light detector 604 decreasing signal to noise ratio by eliminating shallow and noisier signals.

This has been unexpectedly discovered to be a direct result of the narrow beam radiation pattern 626 of less than 30°, which is inherent in VCSEL light sources and directly results in less noise because the shallower layers of the tissue 612 are not measured and therefore the measurements containing large amounts of noise from the stratum corneum 614, the epidermis 616, the papillary dermis 618, the superior blood net dermis 620, and the reticular dermis 621 do not add noise to the measurement obtained from deeper in the tissue 612.

Although the light 610 is not appreciably reflected or back-scattered and absorbed within the shallower layers of the tissue 612 previously described, the light 610 is back-scattered and absorbed within the deeper layers of the tissue 612, and particularly within the inferior blood net dermis 622, for example. The light 610 reflected within the inferior blood net dermis 622 is shown incident on the light detector 604 after the light path 630 traverses through deeper portions of the tissue 612.

It has been discovered that the deeper portions of the tissue 612, such as the inferior blood net dermis 622, provide better measurements as the volume of blood is larger and more active within the inferior blood net dermis 622. Measurements from deeper within the tissue 612 such as within the inferior blood net dermis 622, can therefore provide better measurements with less noise than obtaining measurements from shallower tissue layers.

The light 610 emitted by the light emitter 602 having the narrow beam radiation pattern 626 of the VCSEL reduces noise without increasing power or the separation distance 606 between the light emitter 602 and the light detector 604 and thus provides an appreciable increase in performance. This is due to the light emitter 602 measuring deep within the tissue 612 at the inferior blood net dermis 622 because the radiation pattern 626 is a narrow beam (generally less than 30°, almost always less than 40°), and any reflection from shallower layers of the tissue 612 is not detected by the light detector 604 as the light path 630 is more confined.

Furthermore, use of the light emitter 602 implemented as a VCSEL also provides the benefit of not requiring any elements within the light path 630 to further restrict the light 610 from being measured by the light detector 604, thus reducing costs, part counts, and manufacturing complexity. The light emitter 602, being a VCSEL, provides the light 610 with a narrow beam radiation pattern that is more angularly constrained compared to LEDs and thereby allows measurements of deep within the tissue 612 at a fraction of the power required.

Illustratively, for example, it has been discovered that the light emitter 602 implemented as a VCSEL can provide measurements with better signal to noise ratios while utilizing less than half of the power of a comparable LED light source. This is due to the directed nature of the light back-scattering deep within the tissue 612 rather than from most layers of tissue, which is typical of an LED implementation. The light emitter 602 can also provide multiple different wavelengths of the light 610.

For example, it is contemplated that the light emitter 602 can be a VCSEL having a wavelength of 940 nm, 660 nm, or 530 nm. A collection of all three or any combination thereof, is also contemplated to be an array describing the light emitter 602. The optical sensor system 600 providing a 940 nm light emitter 602 can be used to provide motion data, a 660 nm light emitter 602 can be used to provide SPO2 data, and a 530 nm light emitter 602 can be used to provide heart rate data.

The light emitter 602 and the light detector 604 can be mounted to a sensor body 632 having vertically extended sidewalls 634. The vertically extended sidewalls 634 extend vertically past the light detector 604 and vertically past the top surface 628 of the light emitter 602.

A protective layer 636 shown mounted to the vertically extended sidewalls 634 and extended over both the light detector 604 and the light emitter 602. The protective layer 636 can be glass, polymer, ceramic, or a combination thereof and have an optical transparency in the wavelengths at which the light emitter 602 operates.

The optical sensor system 600 can be operated with the protective layer 636 in direct contact with the tissue 612 or can be operated with a space between the tissue 612 and the protective layer 636 as shown.

Referring now to FIG. 7, therein is shown a cross-sectional view of the light emitter 602 of FIG. 6. The light emitter 602 is shown implemented as a simplified vertical cavity surface emitting laser.

In order to avoid obscuring the optical sensor system, some well-known system configurations and descriptions are not disclosed in detail. For example, the light emitter is shown simplified for clarity. Furthermore, some common configurations of lasers such as substrate emitting VCSELs or VCELs utilizing sapphire substrates are contemplated to be used with the optical sensor system 600; however, because other lasers produce the radiation pattern 626 having a narrow beam, the distinguishing traits of other various lasers is not intended to limit the optical sensor system 600.

The light emitter 602 is depicted having a substrate 702 with a lower reflector 704 formed thereover. The substrate 702 can be formed of gallium arsenide, for example.

The lower reflector 704 can be an n-type distributed Bragg reflector having alternating layers of varying refractive index. Each of the layers within the lower reflector 704 can partially reflect light within the light emitter 602 to form a high quality reflector. The lower reflector 704 can be composed of single crystal layers one quarter wavelength thick.

Above the lower reflector 704, a lower spacer 706 can be formed thereover. The lower spacer can be AlGaAs or AlGaInP, for example. Above the lower spacer 706, an active region 708 can be deposited as a horizontal layer or multiple horizontal layers.

The active region 708 can be a Multi-Quantum-Well (MQW) active region formed of AlGaInP/GaInP or InGaN/GaN, for example. Above the active region 708, an upper spacer 710 can be formed. The upper spacer 710 can be formed of AlGaAs or AlGaInP as the lower spacer 706 is.

The active region 708 can therefore be seen sandwiched between the lower spacer 706 and the upper spacer 710. Above the upper spacer 710, an oxide aperture 712 can be formed. The oxide aperture 712 can contribute to the angularly constrained narrow beam radiation pattern 626 of FIG. 6.

Above the oxide aperture 712, an upper reflector 714 can be deposited. The upper reflector 714 can be a p-type distributed Bragg reflector having alternating layers of varying refractive index. Each of the layers within the upper reflector 714 can partially reflect light within the light emitter 602 to form a high quality reflector. The upper reflector 714 can be composed of single crystal layers one quarter wavelength thick.

Above the upper reflector 714, a metal contact 716 having an upper aperture 718 can be formed. The upper aperture 718 can be vertically aligned with the oxide aperture 712 allowing the light 610 of FIG. 6 to be generated within the active region 708 and to be emitted through the oxide aperture 712 and the upper aperture 718.

It is to be understood that the light emitter 602 can be implemented with different structural elements, such as being formed having the substrate 702 formed as an upper surface from which the light 610 is emitted. However, the light emitter 602 is contemplated to share attributes including the active region 708, which constitutes the “vertical cavity” within the VCSEL.

Referring now to FIG. 8, therein is shown a control flow for the manufacture of the optical sensor system 600 of FIG. 6. The method of manufacture can include providing a sensor body having a vertical extension in a block 802; mounting a laser light emitter to the sensor body, the laser light emitter for emitting a narrow beam of light into tissue in a block 804; and mounting a light detector to the sensor body with the vertical extension between the light detector and the light emitter, the light detector detects the light, the narrow beam of light for providing a physiological measurement from within the inferior blood net dermis 622 of FIG. 6 tissue in a block 806.

Thus, it has been discovered that mechanisms of forming the narrow beam of the light within the optical sensor system increases signal to noise ratio without increasing the separation distance between the light emitter and light detector and without increasing power. The optical sensor system therefore furnishes important and heretofore unknown and unavailable solutions, capabilities, and functional aspects. The resulting configurations are straightforward, cost-effective, uncomplicated, highly versatile, accurate, sensitive, and effective, and can be implemented by adapting known components for ready, efficient, and economical manufacturing, application, and utilization.

While the optical sensor system has been described in conjunction with a specific best mode, it is to be understood that many alternatives, modifications, and variations will be apparent to those skilled in the art in light of the preceding description. Accordingly, it is intended to embrace all such alternatives, modifications, and variations, which fall within the scope of the included claims. All matters set forth herein or shown in the accompanying drawings are to be interpreted in an illustrative and non-limiting sense.

1. An optical sensor system comprising:
a sensor body;
a laser mounted to the sensor body, the laser for emitting light into a cutaneous plexus within a tissue; and
a light detector mounted to the sensor body, the light detector configured to detect the light and provide a physiological measurement from within the cutaneous plexus.

2-5. (canceled)

6. An optical sensor system comprising:
a sensor body having a vertical extension;
a laser mounted to the sensor body, the laser for emitting light into a cutaneous plexus within a tissue; and
a light detector mounted to the sensor body with the vertical extension between the light detector and the laser, the light detector configured to detect the light and provide a physiological measurement from within the cutaneous plexus.

7-10. (canceled)

11. A method of manufacturing an optical sensor system comprising: providing a sensor body;
mounting a laser to the sensor body, the laser for emitting light into a cutaneous plexus within a tissue; and
mounting a light detector to the sensor body, the light detector configured to detect the light and provide a physiological measurement from within the cutaneous plexus.

12-15. (canceled)

16. The method of claim 11 wherein:
mounting the sensor body includes mounting the sensor body having a vertical extension; and
mounting the light detector includes mounting the light detector to the sensor body with the vertical extension between the light detector and the laser.

17-20. (canceled)

21. The system of claim 1 wherein: the laser is a vertical cavity surface emitting laser.

22. The system of claim 1 wherein: the laser is configured to emit the light in a wavelength for providing motion data.

23. The system of claim 1 wherein: the laser is configured to emit the light in a wavelength for providing SPO2 data.

24. The system of claim 1 wherein: the laser is configured to emit the light in a wavelength for providing heart rate data.

25. The system of claim 6 wherein: the sensor body includes a vertically extended sidewall extended vertically past the light detector and vertically past the laser.

26. The system of claim 25 further comprising: a protective layer mounted to the vertically extended sidewall over both the light detector and the laser.

27. The system of claim 6 wherein: the laser includes an oxide aperture configured to provide an angularly constrained narrow beam radiation pattern.

28. The system of claim 27 wherein: the laser includes a metal contact having an upper aperture vertically aligned with the oxide aperture.

29. The method of claim 11 wherein: mounting the laser includes mounting a vertical cavity surface emitting laser.

30. The method of claim 11 wherein: mounting the laser includes mounting the laser configured to emit the light in a wavelength for providing motion data.

31. The method of claim 11 wherein: mounting the laser includes mounting the laser configured to emit the light in a wavelength for providing SPO2 data.

32. The method of claim 11 wherein: mounting the laser includes mounting the laser configured to emit the light in a wavelength for providing heart rate data.

33. The method of claim 16 wherein: providing the sensor body includes providing the sensor body having a vertically extended sidewall extended vertically past the light detector and vertically past the laser.

34. The method of claim 33 further comprising: mounting a protective layer to the vertically extended sidewall over both the light detector and the laser.

35. The method of claim 16 wherein: mounting the laser includes mounting the laser having an oxide aperture configured to provide an angularly constrained narrow beam radiation pattern.

36. The method of claim 35 wherein: mounting the laser includes mounting the laser having a metal contact having an upper aperture vertically aligned with the oxide aperture.
